(19) United States
(12) Patent Application Publication
(10) Pub. No.: US 2019/0380641 A1
(43) Pub. Date: Dec. 19, 2019

(54) SMART BRASSIERE PREVENTING BREAST CANCER AND OTHER BREAST DISEASES IN ADVANCE

(71) Applicant: EVELYN TECHNOLOGY INC, WILMINGTON (DE)

(72) Inventors: HUANSHAN NONG, NANNING (CN); SHOUGANG CHE, SHENZHEN (CN)

(21) Appl. No.: 16/111,404

(22) Filed: Aug. 24, 2018

(30) Foreign Application Priority Data
Jun. 19, 2018 (CN) 201810627912.X

(51) Int. Cl. A61B 5/00; A61B 5/0205; A41C 3/00

(52) U.S. Cl. CPC A61B 5/4312; A61B 5/02055; A61B 5/0402; A41C 3/0064; A61B 5/6804

(57) ABSTRACT

The invention discloses the smart brassiere which is used to prevent the breast cancer and other breast diseases in advance, and it includes two cups. There's arc-shaped sunk inner end face on cups and inner end face is installed with multiple sensors to monitor data information of breasts. There are various sensors inside the cups and they are respectively located in the different areas of the inner end face of the cups. The sensors on inner end face of cups present axisymmetric arrangement with symmetry axis of two cups being the center. Sensors and database system are connected through wireless signal. Through monitoring various data information of breasts with sensors for many times, data information will be transmitted to database system for storage, and the latest data information will compare with old data information to judge possible diseases of breasts.

### TECHNICAL FIELD

The invention is related to technical field of brassiere, especially smart brassiere which is used to prevent breast cancer and other breast diseases in advance.

### DESCRIPTION OF RELATED ART

The mammary gland of female is composed of skin, fibrous tissue, gland and fat. Breast cancer is malignant tumor on epithelial tissue of mammary gland and it's one of major diseases harming physical and psychological health of female. Besides, it's difficult to detect.

During treatment process of breast cancer, lesion location generally needs excision and it brings great psychological harm to the female. As a result, prevention of breast cancer is extremely important.

At present, most of the female find that they at least suffer from breast cancer at an early stage after physical examination in hospital. The examination in hospital can't prevent lesion in advance and it will be treated after detection. Therefore, preventing breast cancer and other breast diseases in advance is a hard nut to crack.

### SUMMARY

Smart brassiere preventing breast cancer and other breast diseases in advance disclosed by the invention hereof aims to solve the problem that breast cancer and other breast diseases can't be prevented in advance by using prior art.

The realization of the invention hereof is as below. Smart brassiere preventing breast cancer and other breast diseases in advance includes two cups wrapping breasts. There's arc-shaped sunk inner end face on cups and inner end face is installed with multiple sensors to monitor data information of breasts. There are many sensors inside the cups and they are respectively located in the different areas of the inner end face of the cups. The sensors on inner end face of two cups present axisymmetric arrangement with symmetry axis of two cups being the center. All sensors and database system are connected through wireless signal.

All sensors include electromyography sensor, sweat sensor, vascular sensor, electrocardiogram sensor, microelectronics sensor, body temperature sensor, pressure sensor, motion sensor, environmental sensor and PH sensor.

There are corresponding papilla points for papilla on inner end face of cups, and each kind of sensor presents spiral arrangement outward by surrounding papilla points.

All sensors are successively arranged adjacently or at intervals. In other words, the intervals between adjacently same kind of sensor are successively arranged with other kinds of sensor.

The cups include outer cover layer and inner cover layer clinging to breasts. Outer cover layer and inner cover layer adopt superimposed arrangement. All sensors are embedded in the interlayer between outer cover layer and inner cover layer.

Inner cover layer has inner interlayer end face towards outer cover. On inner interlayer end face, there's bar-type groove presenting spiral arrangement. All sensors are successively embedded in the bar-type groove adjacently or at intervals.

Bar-type groove is covered by outer cover layer to form bar-type cavity.

All sensors are flexible sensors.

Comparing with prior art, smart brassiere preventing breast cancer and other breast diseases in advance disclosed by the invention hereof monitors various data information of breasts with sensors for many times, and transmits data information to database system for storage. Subsequent monitoring of data information will compare with previous data information to judge possible diseases of breasts. After monitoring works for many times, the accumulation of data information will make the consequent monitored data information more accurate and the judgement become more reliable. Possible lesion of breasts can be judged through the difference of data information, and then make prevention in advance, which solves the problem that breast cancer and other breast diseases can't be prevented in advance by using prior art.

### BRIEF DESCRIPTION OF THE DRAWINGS

FIG. 1 is structure diagram of a smart brassiere preventing breast cancer and other breast diseases in advance provided by an embodiment of the invention hereof.

FIG. 2 is disassembling diagram of the smart brassiere preventing breast cancer and other breast diseases in advance provided by the embodiment of the invention hereof.

### DETAILED DESCRIPTION OF PREFERRED EMBODIMENT

To make the purpose, technical scheme and advantages of the invention hereof clearly understand, further details of the invention hereof are described by referring to accompanying drawings and embodiment. It shall be understood that the foregoing descriptions of the embodiment are intended to illustrate and not to limit the invention hereof.

Further details of realizing the invention hereof is described by referring to following specific embodiment.

In the accompanying drawings of embodiments, same or similar labels are corresponding to the same or similar parts. In the description of the invention hereof, it shall be understood that if any directions or positional relations indicated by terminologies such as “up”, “down”, “left” and “right”, they are based on the directions or positional relations shown by accompanying drawings. They are simply used for convenient and concise descriptions of the invention hereof, but not to indicate or imply that corresponding apparatus or components must possess specific directions, and shall be constructed and operated as per specific directions. Therefore, the words describing positional relations in accompanying drawings are intended for exemplary description, but not to limit the invention hereof. For those skilled in the art, they can understand the specific meanings of above-mentioned terminologies based on specific situations.

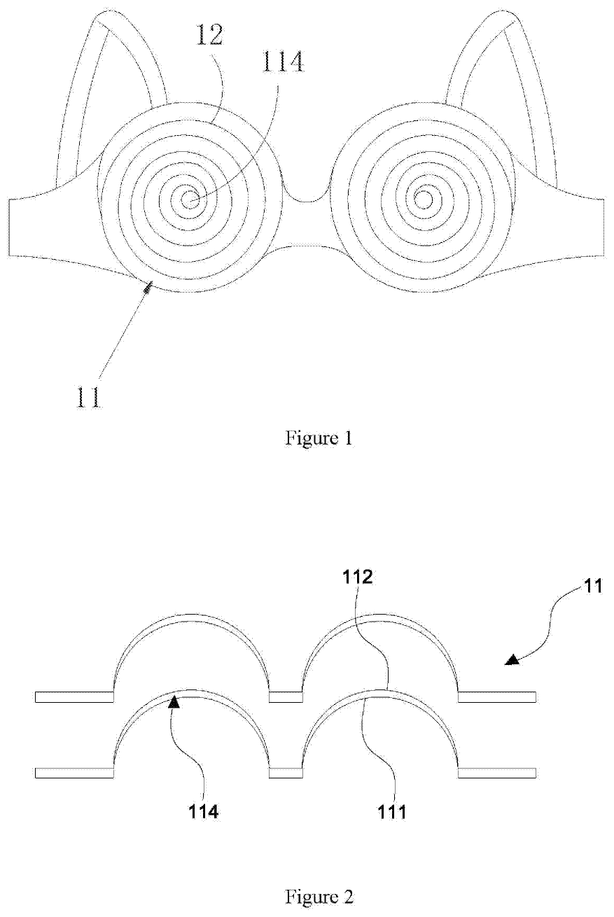

By referring to FIG. 1 and FIG. 2, they provide a good embodiment for the invention hereof.

A smart brassiere preventing breast cancer and other breast diseases in advance provided in the embodiment is used to monitor data information of breasts and compare with previous monitored data information, thus foreseeing the location of possible breast cancer and other breast diseases, receiving health guidance or physical adjustment in advance, reducing the occurrence probability of breast cancer and effectively preventing in advance.

The smart brassiere preventing breast cancer and other breast diseases in advance provided in the embodiment includes two cups 11 wrapping the breasts. There's arc-shaped sunk inner end face on cups 11 and inner end face of cups 11 is installed with multiple sensors 12 to monitor data information of breasts. There are many sensors 12 inside the cups and they are respectively located in the different areas of the inner end face of the cups 11. All sensors 12 on inner end face of cups 11 present axisymmetric arrangement with symmetry axis of two cups 11 being the center. All sensors 12 and database system are connected through wireless signal.

Through monitoring various data information of breasts with sensors 12 for many times, data information will be transmitted to database system for storage, and subsequent monitoring of data information will compare with previous data information to judge possible diseases of breasts. After monitoring works for many times, the accumulation of data information will make the consequent monitored data information more accurate and the judgement become more reliable. Possible lesion of breasts can be judged through the difference of data information, and then make prevention in advance.

Above-mentioned sensors 12 spreading over the inner end face of cups 11 can adequately measure each point location of breasts. After integrating data information of different point locations on cups 11, surface diagram can be made with distribution locations of same kind of sensor 12 on one breast being horizontal coordinate and data information measured by same kind of sensor 12 being vertical coordinate. In general, various data information on breasts presents smooth transition. If any lesion of certain location, the measured surface diagram will present evident convex or concave, from which to judge lesion location on breasts, thus receiving examination or health adjustment promptly.

Symmetrically distributed sensors 12 can compare data information of two breasts respectively measured by two cups 11 to make judgement and prevention in advance.

Common judgement by comparing data information at different time intervals, comparing data information at same time intervals and comparing symmetric data of two cups 11 at same time intervals can increase the accuracy of judgement.

In this embodiment, there's microprocessor on the brassiere. Above-mentioned sensors 12 are electrically connected with microprocessor. In the microprocessor, there are a plurality of power supply components supplying power for sensors. The microprocessor is connected with database system signal to transmit data. Through comparison, analysis & integration by database system, make judgement and transmit judged information to app on mobile for feedback.

The microprocessor is on joint position of two cups 11, namely central position of brassiere, which is convenient for data information transmission of sensors 12.

The microprocessor and database system transmit data through NFC or ultra low-consumption Bluetooth, so it will not produce radiation or be harmful for human body.

Besides, the database system can upload personal data information to cloud-end database. As more and more people use it, a big data will produce in the cloud-end database, which will make it easy to collect data for medical treatment and prevent various breast diseases such as breast cancer in advance.

Cloud-end database will analyze and integrate data information of multiple users to obtain threshold value of health data. Through threshold value of health data, directly judge if any health problem with the breasts of the female, which is simple and fast, and treats breast diseases of the female more rapidly.

When new user wears this brassiere for the first time, database system will compare collected data information with threshold value of health data in the cloud-end database to judge point locations with problems on the breasts of new user, and to remind new user receiving health guidance and treatment promptly. In other words, through constant accumulation of data, when subsequent users use this smart brassiere, it's easier to accurately judge the location with possible health problem on breasts, so that the user can receive health guidance or corresponding physiotherapy promptly.

In the embodiment, above-mentioned sensors 12 include electromyography sensor, sweat sensor, vascular sensor, electrocardiogram sensor, microelectronics sensor, body temperature sensor, pressure sensor, motion sensor, environmental sensor and PH sensor. Electromyography sensor monitors muscular activity of breasts. Sweat sensor monitors diabetes (monitor blood glucose), nephrosis (monitor carbamide and creatinine), thyroiditis (monitor thyroid hormones) and sex hormone, etc. Vascular sensor monitors cardiac function, vascular conditions, blood conditions and microcirculation function. Electrocardiogram sensor monitors an electrocardio signal. Microelectronics sensor monitors micro-current of each point location. Body temperature sensor monitors temperature of each point location. Pressure sensor monitors pressure of each point location on cups 11. Motion sensor monitors micro-motion conditions of each point location. Environmental sensor monitors environment humidity of each point location, etc. PH sensor monitors PH value of each point location. Common coordination and use of sensors 12 reduce measuring error of using single sensor 12. Common function of sensors 12 can judge the position of lesion more accurately. Apart from breast diseases such as breast cancer, it can also monitor other physiological problems.

In the embodiment, there are corresponding papilla points 114 for papilla on inner end face of cups, and each kind of sensor 12 presents spiral arrangement outward by surrounding papilla points 114. Spiral arrangement can fully cover the entire cups 11 to make the monitored data information more complete. Besides, spiral arrangement makes wiring of sensors 12 convenient, so that sensors 12 are electrically connected with a microprocessor.

Besides, it's sunken inward on papilla points 114 of inner end face of cups 11 so that the papilla can embed, which makes the brassiere comfortable to wear.

Sensors 12 are successively arranged adjacently or at intervals. In other words, the intervals between adjacently same kind of sensor 12 are successively arranged with other kinds of sensor 12. In this way, it can avoid inaccurate measurement data caused by superimposition of different sensors 12.

All sensors 12 are flexible sensors, so it's easy to fold as clothes and will not deform while wearing. All sensors 12 are laminar and cling to the cups 11 more tightly. It will not be damaged easily and convenient for cleaning.

In the embodiment, cups 11 include outer cover layer 112 and inner cover layer 111 clinging to breasts. Outer cover layer 112 and inner cover layer 111 adopt superimposed arrangement. All sensors 12 are embedded in the interlayer 114 between outer cover layer 112 and inner cover layer 111. As a result, above-mentioned sensors 12 can be steadily fastened to avoid inaccurate monitored data caused by falling of sensors 12.

To avoid position offset of sensors 12 during using process for a long time, inner cover layer 111 has inner interlayer end face towards outer cover 112. On inner interlayer end face, there's bar-type groove presenting spiral arrangement. All sensors 12 are successively embedded in the bar-type groove adjacently or at intervals. Outer cover 112 is superimposed on inner cover 111, and bar-type groove is covered by outer cover layer 112 to form bar-type cavity, thus making all sensors 12 in the bar-type groove fasten more steadily.

In the embodiment, database system gives feedback about data information measured by sensors 12 to software such as mobile app, etc. Through mobile app, presented surface image model of data information measured by sensors 12 can be seen visually. By comparing with previous data information, point location with health problem will be highlighted to visually judge location with possible lesion, thus receiving health guidance or physiotherapy promptly. Besides, through mobile app, data information can be selectively uploaded to cloud-end database to protect personal privacy of users.

Above is simply good embodiment of the invention hereof, but not to limit the invention hereof. Any modifications, equal substitutions and improvements without departing from the spirit and principles of the invention hereof shall be within protection scope of the invention hereof.

What is claimed is:

1. A smart brassiere preventing breast cancer and other breast diseases in advance, comprising two cups, and the two cups having arc-shaped sunk inner end faces, wherein various sensors are disposed to monitor data information of breasts, the quantity of every kind of the sensors is multiple, the sensors are respectively spread over the inner end face of the cups at intervals, the sensors at the inner end faces of the two cups are symmetrically arranged by taking the symmetry axis of the two cups as the cultural axis, the sensors are connected with a database system through wireless signal.

2. The smart brassiere preventing breast cancer and other breast diseases in advance according to claim 1, wherein the smart brassiere preventing breast cancer and other breast diseases in advance comprises various sensors, namely electromyography sensor, sweat sensor, vascular sensor, electrocardiogram sensor, microelectronics sensor, body temperature sensor, pressure sensor, motion sensor, environmental sensor and PH sensor.

3. The smart brassiere preventing breast cancer and other breast diseases in advance according to claim 1, wherein for the smart brassiere preventing breast cancer and other breast diseases in advance, there are corresponding papilla points for papilla on the inner end face of the cups and each kind of the sensor is spirally arranged outward by surrounding the papilla points.

4. The smart brassiere preventing breast cancer and other breast diseases in advance according to claim 3, wherein for the smart brassiere preventing breast cancer and other breast diseases in advance, all the sensors are successively arranged adjacently or at intervals, namely that the intervals between adjacently same kind of the sensors are successively arranged with other kinds of the sensors.

5. The smart brassiere preventing breast cancer and other breast diseases in advance according to claim 3, wherein for the smart brassiere preventing breast cancer and other breast diseases in advance, the cups include an outer cover layer and an inner cover layer clinging to breasts, wherein the outer cover layer and the inner cover layer adopt superimposed arrangement, while all the sensors are embedded in the interlayer between the outer cover layer and the inner cover layer.

6. The smart brassiere preventing breast cancer and other breast diseases in advance according to claim 5, wherein for the smart brassiere preventing breast cancer and other breast diseases in advance, the inner cover layer has inner interlayer end faces towards the outer cover, ‘there are bar-type groove being arranged on the inner interlayer end faces, while all the sensors are successively embedded in the bar-type groove adjacently or at intervals.

7. The smart brassiere preventing breast cancer and other breast diseases in advance according to claim 6, wherein for the smart brassiere preventing breast cancer and other breast diseases in advance, the bar-type grooves are covered by the outer cover layer to form bar-type cavity.

8. The smart brassiere preventing breast cancer and other breast diseases in advance according to claim 1, for the smart brassiere preventing breast cancer and other breast diseases in advance, all the sensors are flexible sensors.

9. The smart brassiere preventing breast cancer and other breast diseases in advance according to claim 2, for the smart brassiere preventing breast cancer and other breast diseases in advance, all the sensors are flexible sensors.

10. The smart brassiere preventing breast cancer and other breast diseases in advance according to claim 3, for the smart brassiere preventing breast cancer and other breast diseases in advance, all the sensors are flexible sensors.

11. The smart brassiere preventing breast cancer and other breast diseases in advance according to claim 4, for the smart brassiere preventing breast cancer and other breast diseases in advance, all the sensors are flexible sensors.

12. The smart brassiere preventing breast cancer and other breast diseases in advance according to claim 5, for the smart brassiere preventing breast cancer and other breast diseases in advance, all the sensors are flexible sensors.

13. The smart brassiere preventing breast cancer and other breast diseases in advance according to claim 6, for the smart brassiere preventing breast cancer and other breast diseases in advance, all the sensors are flexible sensors.

14. The smart brassiere preventing breast cancer and other breast diseases in advance according to claim 7, for the smart brassiere preventing breast cancer and other breast diseases in advance, all the sensors are flexible sensors.
